(19) United States
(12) Patent Application Publication
(10) Pub. No.: US 2020/0182697 A1
(43) Pub. Date: Jun. 11, 2020

(54) ACTIVE ILLUMINATION 2D AND/OR 3D IMAGING DEVICE FOR AGRICULTURE

(71) Applicant: GroView Pty Ltd, Cremome, New South Wales (AU)

(72) Inventors: Ran Asher Peleg, Cremorne (AU); Mark Julian Blum, Vaucluse (AU)

(21) Appl. No.: 16/624,725

(22) PCT Filed: Jul. 16, 2018
(86) PCT No.: PCT/AU2018/050735

§ 371 Date: Dec. 24, 2019

(30) Foreign Application Priority Data
Jul. 20, 2017 (AU) 2017902843

(51) Int. Cl. G01J 3/28; G01N 33/24; G01N 21/31; G06T 7/00

(52) U.S. Cl. CPC G01J 3/2823; G01N 33/246; G01J 2003/003; G06T 7/0004; G01N 21/31

(57) ABSTRACT

A spectral imaging device for determining a growth stage of a biomass resource. The device comprising one or two (stereoscopic) cameras and an active illumination means emitting a spectral wavelength. The cameras adapted to capture at least two images comprising data captured in the spectral wavelength emittable by the active illumination means. A processor adapted to analyse at least one image; and wherein images captured by stereoscopic camera generates a 2D and/or a 3D image.

### TECHNICAL FIELD

The device of the present disclosure may relate to an image capturing means adapted to monitor a spatial area. More particularly, the device of the present disclosure may relate to an imaging device adapted to monitor a special area using active illumination and the ability to generate a 2D and/or 3D image data.

### BACKGROUND

Farming has become increasingly automated and more precise with the advancements of technology. This has in turn resulted in more sophisticated farming techniques to be developed to monitor and maintain crops and other farming resources. However, there are still some aspects for farming and resource management which still require labour intensive processes or require a skilled person to make a visual assessment to make a determination as to how best utilise present resources.

One of the most important resources for sustaining livestock is a pasture. Typically, livestock can generally be sustained on pasture resources alone. However, determining whether to let a pasture rest or whether a pasture contains a sufficient amount of biomass for livestock to utilise is difficult and generally requires a skilled visual assessment by a farmhand or other person. This method is highly subjective and can lead to a number of significant problems at later times if the assessment is incorrect, such as loss of livestock or crop resources.

Crop monitoring is also an essential component of farming biomass resources as the crops will progress through growth stages and experience different weather conditions. During growing of crops, pesticides and fertiliser will be typically needed to improve the potential for yield of biomass resources. However, monitoring of crops generally will be done by visual assessment by persons monitoring the crops, and may result in premature or late application of pesticides and/or fertiliser, which can result in excess pesticides and/or fertilisers being applied to the crops.

There are also a number of known devices which may be used to monitor crops, such as drones. These devices are generally expensive and impractical for use to monitor growth of biomass resources, and still heavily rely on visual interpretation by a skilled person to assess images captured.

Satellites may also be used to monitor an area, however these are expensive tools to use and are dependent on global positioning and/or are limited by adverse weather conditions which may mask or conceal a pasture or location desired to be viewed. Further, satellites are not well suited to determining soil conditions, or viewing more than a top view of a location which would make calculation of a biomass or growing resource virtually impossible.

Any discussion of the prior art throughout the specification should in no way be considered as an admission that such prior art is widely known or forms part of common general knowledge in the field.

### SUMMARY

### Problems to be Solved

It may be advantageous to provide a means for allowing remote monitoring of organic assets, such as crops, such as wheat, cotton, corn etc.

It may be advantageous to provide a means for allowing remote monitoring of pastures or crops.

It may be advantageous to provide a device for recording data in relation to pastures or crops.

It may be advantageous to allow for periodic monitoring of farmland for agricultural use.

It may be advantageous to provide for a device which is adapted to monitor for growth stages of vegetable, fruit and/or nut plants.

It may be advantageous to provide for a device which can identify when fertilizers and/or pesticides are required to be provided to crops and/or orchards.

It is an object of the present invention to overcome or ameliorate at least one of the disadvantages of the prior art, or to provide a useful alternative.

### Means for Solving the Problem

In one aspect of the present disclosure, there is provided a spectral imaging device for determining a growth stage of a biomass resource. The device comprising one or two (stereoscopic) cameras and an active illumination means for emitting various spectral wavelengths. The cameras are adapted to capture at least one image comprising data captured in the spectral wavelength emitted by the active illumination means. A processor adapted to analyse the at least one image; and wherein the captured two images of the stereoscopic camera generates a 3D image.

Preferably, the active illumination means comprises at least one LED. Preferably, a biomass index value can be generated with respect to the captured 2D or 3D images. Preferably, the device further comprises a communication means to transmit data. Preferably, the device further comprises a power source in communication with a renewable energy generator. Preferably, the device comprises a housing in which the image capture means is stored. Preferably, the device is adapted to record and store at least one 2D or 3D image. Preferably, two 2D or 3D images and a time period between said two images can be used to calculate a growth rate of a biomass resource, however any number of images can be used and with respect to a given time period. Preferably, a plurality of wavelengths are emitted by the at least active illumination means in the range of 200 nm to 1000 nm. Preferably, the device detects the height and/or shape of a biomass resource. Preferably, the device is fixed to a pole and said device can rotate relative to said pole. Preferably, carotenoid pigment levels can be captured such that chlorophyll content of a biomass resource can be detected. Preferably, a Nitrogen content of a biomass resource can be detected. Preferably, the device further comprises a probe adapted to detect the moisture content of soil. Preferably, biomass resource specific features can be detected by the device using active illumination spectroscopy. Preferably, biomass resource feature is selected from the group of; a biomass resource height, a biomass resource growth stage, a number of shoots, plant attributes, flower attributes, fruit attributes, chlorophyll content, sugar level, acidity, vitamin C level, firmness, nitrogen content, and a colour of the biomass resource. Preferably, at least one of satellite data and drone data can be used to augment data captured by the device. Preferably, data captured by the device is transmitted to a server for analysis and presentation to a user. Preferably, a growth rate of a biomass resource is determined with respect to at least two 2D and/or 3D images captured with respect to the time period between the 2D and/or 3D captured images. Preferably, the probe and in combination with the cameras analysis of biomass resource growth stages and calculation of vegetation indices is used to predict biomass, crop yield and biomass resource requirements.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 illustrates a schematic of an embodiment of the device with a spectral image capture means;

FIG. 2 illustrates a schematic of a further embodiment of the device with a stereoscopic spectral image capture means;

FIG. 3 illustrates an embodiment of the image capture means for use with the device of the present disclosure;

FIG. 4 illustrates an embodiment of the image capture means with a plurality of wavelength emission means;

FIG. 5 illustrates an embodiment of an isometric view of an image capture means with a lens cover;

FIG. 6 illustrates an embodiment of an image capture device with a lens cover in a raised position;

FIG. 7 illustrates an embodiment of a multi-spectral projector with an array of light emitting means;

FIG. 8 illustrates an embodiment of a flowchart for processing captured images;

FIG. 9 illustrates an isometric view of another embodiment of an image capture device of the present disclosure with a lens cover down;

FIG. 10 illustrates an isometric view of the embodiment as shown in FIG. 9 with a lens cover up exposing the sensors; and

FIG. 11 illustrates a sectional side view of the embodiments of FIG. 9 with internal electronics mounted therein.

### DESCRIPTION OF THE INVENTION

Preferred embodiments of the invention will now be described with reference to the accompanying drawings and non-limiting examples.

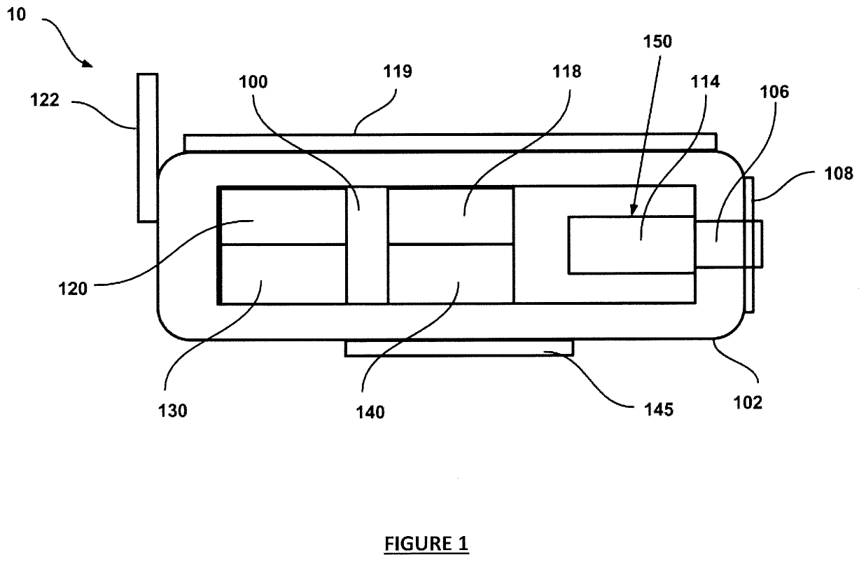

Referring to the embodiment of FIG. 1, a device 10 is shown which comprises a housing 102. The housing 102 may house at least a portion of an image capture means 104, a processor 114 or microcontroller 114, a power source 118, a communication means 120, and a printed circuit board (PCB) 101. The PCB 101 may be used to connect at least one of the image capture means 104, communication means 120, and/or the power source 118. The image capture sensor 150 preferably comprises a sensor 150, a lens 106 and at least one wavelength emitting device 108. The wavelength emitting device may be an array of LEDs and lasers.

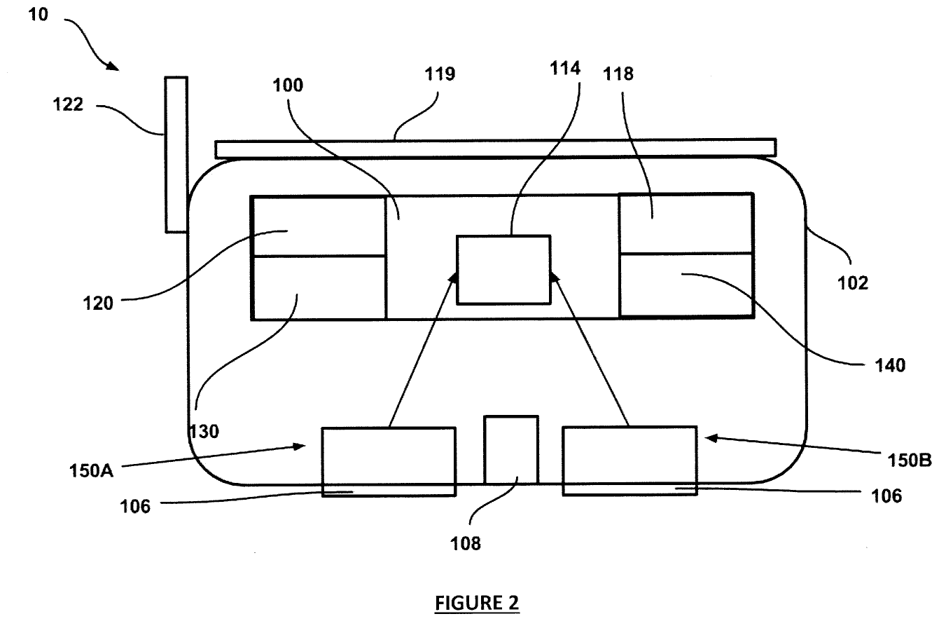

The image capture means 104, which may be an area array detector, digital camera or other image capture means used to detect the intensity of light. However, in a preferred embodiment, the image capture sensor 150 is a stereoscopic camera as can be seen in FIGS. 2 and 6, and has two image capture sensors 150A and 150B. The image capture sensor 150 can allow for capture of wavelengths reflected from the surface of a biomass resource. The array detector may be adapted to function as a charge-coupled device (CCD), a complementary metal oxide semiconductor (CMOS) sensor, and/or an Indium gallium arsenide (InGaAs) sensor for hyperspectral imaging systems. Image capture sensors 150A and 150B may be used to capture and generate data to be used for 3D imaging. The wavelength emitting device 108 is preferably an active illumination means, and optionally a laser 128. Preferably, the active illumination means 108 is positioned between each image capture sensors 150A, 150B of the stereoscopic camera. The stereoscopic camera (sensors 150A, 150B) can be used to generate a three dimensional (3D) image of a biomass resource. A 3D image may be used to determine at least one of height, shape or any other feature of a biomass resource. Preferably, the device 10 is used to monitor crops. The device 10 can be used to monitor a pasture or a field. The device 10 may recognise species of biomass resources or other organic object. Preferably, the device 10 can determine the species of biomass resources by capturing images and comparing the images to a database of known biomass resource species or via spectral signature detection.

As light propagates into a biomass resource, photons are absorbed or scattered. The photons, which are backscattered and exit from the biomass resource in an area (scattering area), illuminate a portion of the biomass resource tissue surrounding the incident area. In this way a spectral signature can be obtained or confirmed. The spectral signature of a biomass resource may also change during growth, and may be indicative of a growth stage change when a spectral signature change is detected with respect to a previous spectral signature captured.

Complete scattering information a biomass resource may be well represented by a single line scan passing near to the centre of the incident area. Having the single line pass near to the centre, rather than at the centre, can improve data collected as signal saturation can be avoided. Although it will be appreciated that the single line can be at the centre.

A biomass resource may include a harvestable resource, such as; a crop, vegetation, fruit, nuts, vegetables, grass, algae or any other photosynthetic organism. It will be appreciated that the biomass resource may include a portion of an organism which is not harvestable, such as a plant from which a consumable can be obtained. As such, a biomass resource may collectively refer to a plant and a harvestable resource thereon, or may refer to a plant without a harvestable resource as the resource has been harvested, or the growth stage of the plant has not yet yielded a harvestable resource. This is of particular advantage as the device is adapted to monitor a growth stage of a biomass resource, so as to predict when a harvestable resource may be about to grow or is growing.

Active illumination may comprise intensification technology with an active source of illumination in the UV, Visible, near infrared (NIR) or shortwave infrared (SWIR) bands. Active infrared illumination may use illumination of spectral range 200 nm to 1,200 nm with cameras sensitive to this light. As active illumination systems can incorporate illuminators that produce high levels of light, the resulting images are typically of a high resolution. Range gated imaging is another form of active illumination which utilises a high powered pulsed light source for illumination and imaging. Range gating is a technique which controls the laser pulses in conjunction with the shutter speed of the image capture means 104 detectors. Gated imaging technology can be divided into single shot, where the detector captures the image from a single light pulse, and multi-shot, where the detector integrates the light pulses from multiple shots to form an image.

The device 10 may comprise at least one motor means 140 to effect movement of the device 10. The motor means 140 may allow for 360 degree rotation of the device 10. The motor means 140 may cause rotation of the device 10 about a vertical axis or may allow for adjustment of the orientation of the pitch and yaw of the device 10. It will be appreciated that the motor means 140 may allow for any predetermined movement of the device 10 such that the device can be directed to target predetermined spatial areas or target locations. Optionally, a photovoltaic cell 119 or other renewable energy collection device may be disposed on the device 10. The photovoltaic cell 119 may be adapted to charge the battery or power source 118. It will be appreciated that the photovoltaic cell 119 may be referred to as a “solar cell” or energy collection means. The solar cell 119 may be disposed on the upper surface of the device 10, or disposed to face a direction which is likely to be the most efficient in relation to collection of energy.

In one embodiment, the device 10 is adapted to determine at least one of; a height of a biomass resource, the species of a biomass resource, the colour of a biomass resource, the number of shoots of a biomass resource, a density of vegetation, or any other desired biomass resource attribute. More preferably, the device 10 is adapted to determine the colour of a biomass resource via spectral imaging. Spectral imaging may be multispectral imaging and/or hyperspectral imaging. The device may also be adapted to distinguish and identify harvestable resource on a biomass resource. Any known spectral imaging techniques and systems may be used by the device 10. The spectral imaging means may be provided without wavelength filters and processing of captured data can remove undesired data to reduce the size of the data collected, and omit irrelevant data sets.

Preferably, the device 10 uses active illumination spectral techniques to capture images of a biomass resource, such as a crop. Spectral range methods of active illumination may be adapted to sense radiation that is invisible to a human observer (i.e. wavelengths in the visible light spectrum). The enhanced spectral range allows the viewer to take advantage of non-visible sources of electromagnetic radiation (such as near-infrared or ultraviolet radiation). Preferably, the device 10 can be fitted with an image capture sensor 150 adapted to detect at least one wavelength in the spectral range. In another embodiment, the image capture sensor 150 can be used as a multispectral image capture means, and/or a hyperspectral capture means.

Intensity range methods may also be used which can be used to detect small quantities of light. If this method is used, the lens of the image capture sensor 150 may be relatively larger to allow for capture of light, as per the inverse square law. An enhanced intensity range may be achieved via technological means through the use of an image intensifier, or other very low-noise and high-sensitivity array of photodetectors.

The volume of biomass resources can be calculated by the device 10 with respect to a weight or a volume. Attributes of a biomass resource may comprise at least one of; a colour of the biomass resource, a height of the biomass resource, a width of the biomass resource, a growth rate of the biomass resource, a growth stage of the biomass resource, a morphology analysis, a spore count or any other predetermined parameter or property of a biomass resource. The growth rate of the resource may be determined by 3D imaging and/or spectral imaging. Such attributes are captured and identified by the capture sensor 150.

Spectral imaging may be at a time when minimal interference is likely to occur, such as a period during the night in which the impact of the sun (and associated wavelengths) is minimised. It will be appreciated that images may be recorded during the day time (hours between sunrise and sunset) to allow for a visual assessment of a biomass resource which can be used in conjunction with at least one spectral image and assessment thereof, such that calculated biomass values can be confirmed by a person.

In one embodiment, the device 10 can calculate a biomass value in a resource location. A biomass value may relate to the volume of a harvestable resource and/or a weight of a biomass resource and/or a harvestable resource. The resource location may be, for example, a field or a pasture which contains biomass resources.

Calculating the biomass value of a location is essential for livestock as the biomass value may be used to calculate a time period for which livestock will be able to survive at said location. Preferably, the time period is in relation to a sustainability time period such that livestock can be moved before consuming an unsustainable amount of biomass which can lead to longer pasture resting times. A pasture resting time may be the time in which a pasture must be free, or substantially free of livestock to return to a viable biomass value to sustain livestock.

The device 10 may be used to monitor a rate of growth of a biomass resource. Monitoring growth can be used to predict a time period for which a pasture can be rehabilitated or return to a desired biomass value. Monitoring growth may also determine a period of time in which the greatest change in a biomass value can be expected before declining. For example, a pasture may have a period of 14 days in which 10 tonnes of biomass is obtained, however the period of 14 days after the initial 14 day period may only generate a further 6 tonnes of biomass. Therefore, as the most biomass is obtained in a period of 14 days, it may be advantageous to move livestock more frequently to allow for consumption of the highest period of growth and allow resting and growth of a previous location to again allow for the highest yield of biomass per time period. The device 10 may also be adapted to calculate a projected biomass value based on a period of time, in combination with at least one of an expected rainfall, an expected temperature and/or historical data. For example, this may allow for a prediction of a biomass value based on the time of year with respect to recent and/or expected rainfall.

Optionally, the device 10 may be adapted to calculate a number of days a predetermined number of livestock can be retained at a location without damaging the sustainability of the resources of the location. For example, if a pasture is to retain thirty (30) cattle, the calculated biomass value may be divided by the number of cattle to obtain a sustainable for biomass value which can be used to determine the number of days that the cattle may be retained at the location without irreparably damaging the biomass resources (for example overconsumption of available grass). The device 10 may then be adapted to issue a message or warning for a resource management worker to move cattle by a predetermined time such that resources can be used more sustainably if possible and with the least amount of effort on behalf of a resource management worker. Similarly, the device 10 may be adapted to calculate the number of days until a predicted growth stage.

The calculation of a vegetation index may be applicable when the device is monitoring a pasture or other area with organic material to be assessed. Vegetation Indices (VIs) are combinations of surface reflectance at two or more wavelengths designed to highlight a particular property of vegetation. They are derived using the reflectance properties of vegetation. Each of the VIs is designed to identify a particular vegetation property.

The indices may be grouped into categories that calculate similar properties. The categories and indices which may be determined by the device 10 may include at least one selected from the following group; Broadband Greenness, Normalized Difference Vegetation Index, Simple Ratio Index, Enhanced Vegetation Index, Atmospherically Resistant Vegetation Index, Sum Green Index, Narrowband Greenness, Red Edge Normalized Difference Vegetation Index, Modified Red Edge Simple Ratio Index, Modified Red Edge Normalized Difference Vegetation Index, Vogelmann Red Edge Index 1, Vogelmann Red Edge Index 2, Vogelmann Red Edge Index 3, Red Edge Position Index, Light Use Efficiency), Photochemical Reflectance Index, Structure Insensitive Pigment Index, Red Green Ratio Index, Canopy Nitrogen, Normalized Difference Nitrogen Index, Dry or Senescent Carbon, Normalized Difference Lignin Index, Cellulose Absorption Index, Plant Senescence Reflectance Index, Leaf Pigments, Carotenoid Reflectance Index 1, Carotenoid Reflectance Index 2, Anthocyanin Reflectance Index 1, Anthocyanin Reflectance Index 2, Canopy Water Content, Water Band Index, Normalized Difference Water Index, Moisture Stress Index, and Normalized Difference Infrared Index.

The VIs that may be calculated on a specific datasets which are determined by the spectral bands detected by the device. If all spectral bands required for a specific index are available, that VI can be available for the dataset. For example, an input dataset from a sensor that matches only the near-infrared and red spectral bands (such as AVHRR, TM, and others) is only able to calculate two of the indices: the NDVI (Normalized Difference Vegetation Index) and SR (Simple Ratio). In contrast, for a high spectral resolution input dataset, such as AVIRIS, twenty-five (25) of the indices may be available. It will be appreciated that any vegetation index may be determined by the device if the device comprises the appropriate active illumination or spectral wavelength emitting means.

The Photochemical Reflectance Index (PRI) is a reflectance measurement. The PRI is sensitive to changes in carotenoid pigments (for example, xanthophyll or chlorophyll pigments) in foliage. Carotenoid pigments are indicative of photosynthetic light use efficiency, or the rate of carbon dioxide uptake by foliage per unit energy absorbed. This may be used to assess vegetation productivity and/or stress. As PRI measures biomass resource responses to stress, it can be used to assess general ecosystem health, which may be useful in assessing health, and therefore a biomass value, of at least one of; vegetation health in evergreen shrublands, forests, and agricultural crops. PRI may be defined by the following equation using reflectance (ρ) at 531 and 570 nm wavelength:

PRI=(ρ531−ρ570)/(ρ531+ρ570)

It will be appreciated that there are variations to the above equation which allow for PRI values to be within the range of −1 to 1, depending on the formulae used. Further, a ratio of foliage of a biomass resource with respect to a harvestable resource may also be calculated.

Normalized Difference Vegetative Index (NDVI) may allow for calculation of an estimated ground coverage, typically by using optical sensors to measure. NDVI is based on measurements of biomass resource reflectivity at different wavelengths:

NDVI=r NIR−(r VIS r NIR)+r VIS

where rNIR is infrared (e.g. 780 nm) reflectivity and rVIS red (e.g. 660 nm) reflectivity. Vigorous biomass resources absorb red and reflect infrared, leading to high NDVI readings. It will be appreciated that other formulas or data sets may be used to generate vegetation values.

The data for calculating the NDVI can be detected by arrays of sensors 150, such as the sensors 150 of the image capture means 104. The sensors 104 are adapted to detect and capture images based on a desired wavelength which may be in the range of ultraviolet and infrared, for example.

Hyperspectral imaging or active illumination image capture can be used to determine the health of a biomass resource and may be used to determine whether there are symptoms of disease present, which can allow a farmer to put measures in place to save biomass resources. Diseases or damage may also alter the spectral signature of a biomass resource. If an unexpected spectral signature change is detected, the device 10 may issue an alert to a user to determine whether the signature change is due to disease, damage or another adverse condition.

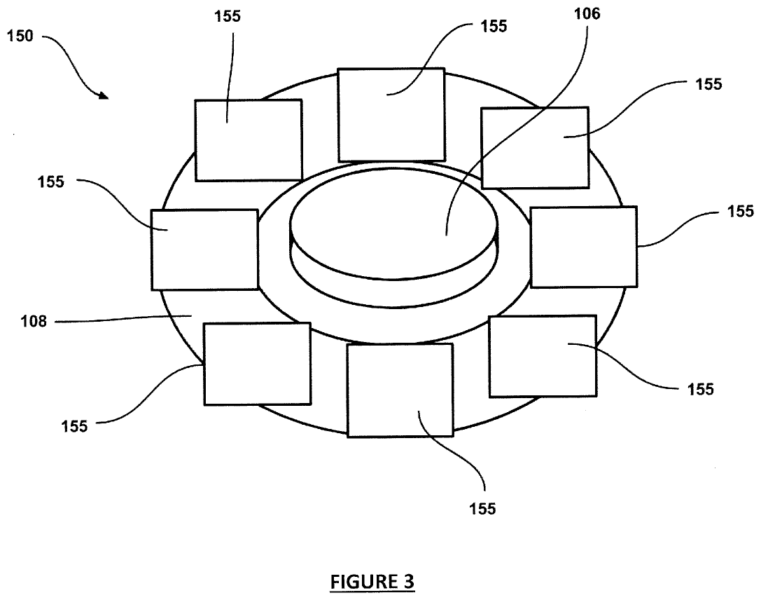

FIG. 3 illustrates an embodiment of an image capture sensor 150. The image capture sensor 150 comprises a lens 106, a plurality of wavelength emitting means 108. Further, LEDs 155 or additional wavelength emitting means can be disposed on the rings 108. A collimater or lens 106 of the image capture sensor 150 may project at least partly beyond the wavelength emitting means 108, such that the wavelength emitting means 108 is positioned between the distal end of the lens and the housing 102. It will be appreciated that the distal end of the lens 106 is the furthest portion of the lens 106 from the housing 102.

The wavelength emitting means 108 are illustrated as LED rings 108. The LED rings 108 may emit predetermined electromagnetic wavelengths in a focus direction of the device 10. The image capture sensor 150 may be used to generate a hyperspectral image to calculate a biomass value. Each ring may have any number of LEDs which may be provided with any desired number of wavelengths.

The LEDs rings 108 can be activated in any predetermined order, or activated at the same instance. The order of activation of the LEDs can be used to manipulate the images captured by the image capture sensor 150. Preferably, a first LED is activated and an image is captured by the device 10, then a second LED is activated and a further image is captured. Remaining LEDs can be activated and images may be captured for each wavelength desired. The captured images may then be combined to be segmented and at least one value assigned to a segment of the image.

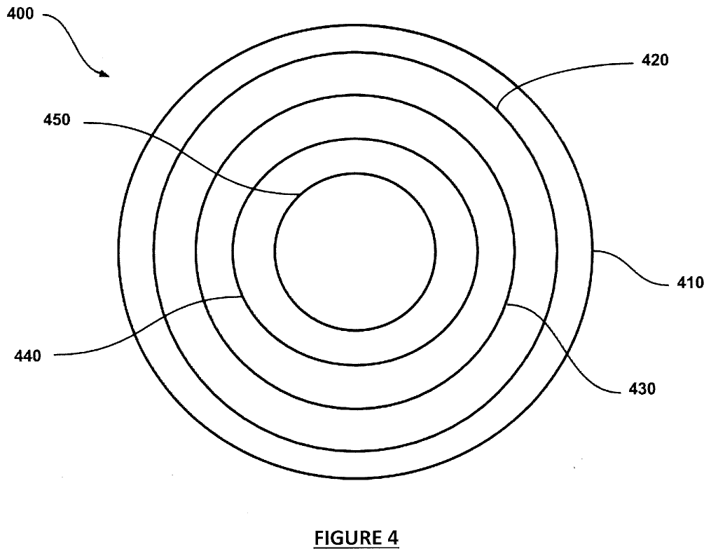

FIG. 4 shows an embodiment of a wavelength emitting means 400 in which a plurality of wavelength emitting means 410, 420, 430, 440, 450 are disposed concentrically. Wavelength emitting means 400 may be used in place of the active illumination means or the wavelength emitting means 108. It will be appreciated that any predetermined array or shape of LEDs 410, 420, 430, 440, 450 may be used with the device 10 of the present disclosure. The wavelength emitting means 410, 420, 430, 440, 450 may be adapted to generate wavelengths in the range of 1 nm to 1200 nm. In a preferred embodiment, the wavelength emitting means 410, 420, 430, 440, 450 is a plurality of light emitting diodes (LEDs) adapted to be used to generate wavelengths of approximately 350 nm, 531 nm, 660 nm, 640 nm and 850 nm. These wavelengths may be varied as desired. It will be appreciated that these wavelengths are exemplary only and are not intended to be limiting. The wavelength emitting means may be adapted to emit wavelengths ranging from ultraviolet to infrared, or near infrared wavelengths.

Optionally, specific wavelengths can be used to allow for the image sensor to detect of carotenoid pigments (for example, xanthophyll or chlorophyll pigments) in foliage. The wavelength emitted is preferably around 350 nm to allow for detection of a chlorophyll florescence.

The communication means 120 may comprise an antenna 122 or aerial such that the communication means can function as a transponder or transceiver which may allow for communication between the device 10 and a further device 10 or a computer or other predetermined electronic device. The device 10 may further comprise a communications means 112 or transceiver adapted to record a reading and relay the reading to a further device, such as a computer, laptop, tablet, mobile phone, cellular phone, smart phone or any other suitable device which may display data sets to a user.

The device 10 may further comprise an accelerometer and/or a gyroscope such that movement of the device 10 can be detected. Detecting movement of a device 10 may be used to assist with determining whether the device 10 is being interacted with, for example if an animal has impacted the device 10 or when there is an unexpected movement of the device 10. If the device 10 moves from a first position to a second position, an alert may be triggered to be sent to a computer or other predetermined device. A movement threshold may also be required to be surpassed such that wind or other minor movements of the device 10 do not issue an alert. An alert may generate a message, a sound, a notification, a beacon, an LED, a light or any other alert which may notify an operator that a device 10 has been moved. In a yet further embodiment, the device 10 is activated once sufficient movement is detected such that at least one image is recorded. Recording an image or issuing an alert may provide a security feature for the device to avoid tampering. The accelerometer can also be used for determining the tilt and roll angle of the device 10.

Image data can be recorded by the image capture sensor 150 and stored in at least one of internal memory, a cloud, an external memory, a second device associated with storage means or any other suitable storage means for retaining data. Optionally, the device 10 may be adapted to record audio or video via the image capture sensor 150.

The image capture sensor 150 of the device 10 can be any predetermined device 10 suitable for recording at least one of; an image, a photograph, a series of images, a video, audio data, a coordinate system, and an array of pixels or the like. While the image capture sensor 150 can have a relatively high pixel (MP) count, such as 5 MP count or greater, the device preferably comprises a image capture means which has less than 5 MP count, and even more preferably comprises a image capture means with a 1 MP count. A reduction in the MP count of the image capture sensor 150 and/or a low spatial resolution (angular resolution) may reduce power consumption and/or storage capacity required by the device 10. The image capture sensor 150 may be adapted to have the minimum MP count and/or resolution for interpreting values on a gauge. As such, the image capture sensor 150 may be preset (pre-set) with a desired resolution and/or MP count, or may be adapted to dynamically reduce or increase the MP count and/or resolution to record a gauge reading. This may ensure that the minimum, or near to the minimum resolution and/or MP count for recording a readable reading image, is recorded.

A mounting means is preferably releasably attached to the device 10. The mounting means (not shown) is adapted to mount onto a post, a pole or other structure. The mounting means maybe a substantially rigid or resilient frame or support which retains the device in a desired location. Alternatively, the mounting means (panning plate 145) may be attached to the housing 102, which may include a frame, and substantially surrounds, encloses or protects at least a portion of the device 10. Having a housing 102 may protect the device 10 from weather or environmental conditions, which may be of particular advantage in an industrial location. Preferably, the housing 102 is water proof and/or water resistant. Preferably, the device 10 comprises an image capture means 104 which acts as a multispectral image capturing means or a hyperspectral image capture means which may be adapted to record images with wavelengths in the range of 200 nm to 1,200 nm. More preferably, the image capture means may capture wavelengths in the range of 300 nm to 1000 nm. Even more preferably, the image capture means may capture wavelengths in the range of 350 nm to 950 nm.

In one preferred embodiment, the hyperspectral image capturing means uses active illumination in various wavelengths. The desired wavelengths are approximately 530 nm, 660 nm, 720 nm and 850 nm. These desired wavelengths may be referred to as green, red, red edge and near infrared. The device might use additional wavelengths as well such as ultraviolet and blue.

The image capture means 104 of the device preferably comprises a wavelength capture sensor 150 and/or wavelength emitting means 108 to emit a plurality of wavelengths which may be inside the visible light spectrum, but preferably also in the near infrared spectrum, and/or in the ultraviolet spectrum. Preferably, the device 10 uses active illumination imaging techniques. In this way the device 10 may be adapted to view light which is not able to be viewed by conventional pasture or crop observation methods and devices. Preferably at least one peripheral sensor 130 means may be installed in the housing 102. The at least one peripheral sensor 130 may be adapted to detect moisture content of soil, detect atmospheric conditions, temperature, humidity or any other desired predetermined parameter to be sensed or detected. The peripheral sensor may assist with determination of biomass resource stress which may be due to water shortage, damage or disease.

Optionally, the image capture means 104 may include hyperspectral imaging means. If hyperspectral imaging means are used with the device, the image capture means may adopt a push broom scanner process which allows for continual reading of an image (or spatial area), or a snapshot imaging process which captures a single image at one time. Hyperspectral imaging and multispectral imaging may utilise a plurality of wavelengths, and preferably uses around five wavelengths, but may use any predetermined number of wavelengths.

The housing 102 may be adapted to be opened and closed by a farmer to access the interior of the housing. Accessing the interior of the housing 102 may be desired if the device 10 requires maintenance or to extract an internal memory of the device. Internal memory of the device 10 may record and store a log of data. The data stored may be at least one of an image, a dataset, a reading, a detected property, errors, error logs, or any other predetermined data set. Preferably the data set can be stored and retained by the device 10 for extended periods of time such that there is a backup of data which is stored away from a computer or network the data is to be sent to. This may allow for determinations of whether the data communicated to the computer or network is accurate by comparing the communicated data records with the stored data records.

The communication means 120 of the device 10 may be adapted to send and receive data. Receiving data may be advantageous as this may allow for remote updating of protocols, activation times, monitoring methods, sunset times, weather conditions, LED activation sequences, updating firmware or any other use for storing data. It will be appreciated that if adverse weather conditions are likely to occur, the device may be adapted to remain in a sleep mode to reduce the potential for damage to the image capture means 104.

In yet a further embodiment, the device 10 may further comprise at least one probe or sensor adapted to determine moisture content of a localised portion of soil. The probe may also be used to determine moisture content of soil. An estimate of the biomass resource of a location may be based on the moisture content detected, or may be used to determine a projected or expected biomass yield of a location. Further, the device 10 may be used to detect at least one of nitrogen and/or phosphate content of a biomass resource or the expected nitrogen and/or phosphate content of soil. Preferably, a nitrogen content of soil can be determined by hyperspectral imaging.

In another embodiment, the device 10 may be used to monitor a pasture, a filed, a location, an area, greenhouse, a barren location, livestock, a reduced atmosphere environment, or any other space which may require monitoring. However, a predominant use of the device is to monitor crops or other agricultural spaces or locations.

The device 10 may have an internal storage means which has plant recognition software to determine a species of biomass resource, or a stage of a biomass resource development cycle and whether a harvestable resource is on a biomass resource. This may be of particular use when determining when to apply fertiliser, chemicals, pesticides, whether a biomass resource has signs of disease, or other additives to the biomass resource and/or soil. At least one growth stage of a biomass resource development cycle yields a harvestable resource.

In this embodiment, the device 10 may determine a height and/or a colour of crops based on detected electromagnetic radiation, preferably an active illumination. The device 10 may comprise a stereoscopic camera which may be used for 3D mapping or 3D imaging. By comparing two images captured by the stereoscopic camera, the two image capture points can be compared to generate a 3D image by conventional methods. 3D mapping or 3D imaging may allow for a determination of at least one of height, shape and/or growth cycle of a viewable biomass resource, animal or object. The device 10 may further be adapted to calculate the number of shoots, tillers, size of a vegetable, size of a fruit, size of a harvestable resource, or other growths on an organic product (such as flora). For example, it may be advantageous to count the number of tillers on wheat or the number of biomass resource growths. The device 10 may further be used to monitor animals or any livestock which may be in a location or spatial area to be monitored.

Using NIR allows capture of shades of red which can be used to render a true colour of an object. This colour rendering or colour processing may optionally be achieved by the device 10 locally without being transmitted or transferred to a computer or other storage means. Electromagnetic wavelengths may have known detectable colours which are associated with known visual light spectrum colours. As such, any predetermined wavelength may be emitted and/or detected by the device 10 to capture and render an image in a desired colour or colours. The device 10 may be adapted to take images during the day, but preferably most images are captured at night or twilight. Images may be captured at any time of the day if desired. Preferably, the device does not require calibration, or can self-calibrate.

Preferably, the wavelength emitting means 108 emits at least one fixed electromagnetic wavelength, and reflectance of the fixed wavelength may be captured by the image capture sensor 150. The device 10 may be adapted to detect moments or instances in which ambient light levels are within a desirable threshold and activate to capture an image in said instance or moment. In this way a more consistent capture method and monitoring may be provided by the device 10. For example, the instance or moment in which ambient light, which may be ambient visual light viewable by a human eye, may be of a lower level due to cloud cover which may provide for an advantageous period, moment or instance to capture an image.

Light reflectance is used to measure the light that is diffusely reflected primarily from the upper or superficial region of a biomass resource. Optionally, visible light in the region of 400 nm to 675 nm can be used for assessing surface features of a biomass resource, and red/NIR light in the region of 675 nm to 1000 nm can be used for penetration and internal quality assessment of a biomass resource. In one embodiment, the device 10 the capture means acquires images using hyperspectral reflectance (400 nm to 675 nm) and transmittance (675 nm to 1000 nm) images simultaneously using one imaging system.

Line scanning can be used for acquiring reflectance. The device 10 may be used for scanning speed and spatial resolution. The acquired raw hyperspectral image needs to be split into two separate images corresponding to each lane. If the split images is “blank” indicating no sample is seen, it will be removed from analysis, or if the image is not blank, data can be extracted from the image.

Optical filters may be omitted by the device 10 which provides a significant advantage with respect to other known hyperspectral devices as the filter is typically a necessity to be able to capture an image and/or data. Without optical filters the device is more economical to manufacture and more easy to maintain in use. Further, omission of the optical filters can allow for an electromagnetic wavelength to be detected and recorded successfully during relatively dark periods of time, such as at night.

Other applications for the device may include land reclamation, land restoration, or mine reclamation. The device may be used to more effectively determine successful plants or ground stabilisation material via monitoring. This may allow installation of the least amount of plant cover to maintain a desired stability, particularly on slopes or mounds of refill material or imported land reclamation material. Further, land degradation or erosion may be recorded during restoration or rehabilitation and may allow for remedial works to be conducted which may assist in reducing rehabilitation fees over a long term period and may also be used to accelerate rehabilitation.

In yet another embodiment, the device 10 may comprise a first and a second capture means. The first capture means and second capture means may allow for depth perception or 3D modelling. An electromagnetic emitting means may be disposed between the first capture means and second capture means. Preferably the electromagnetic emitting means is disposed equidistant between the two first capture means and second capture means. The capture means is preferably a camera or other optical capture means adapted to capture and allow for storage of at least one image containing at least one detected electromagnetic wavelength. For example, at least one detected electromagnetic wavelength is infrared, near infrared, visible light, ultraviolet light or any other predetermined wavelength preferably between the ranges of 100 nanometres (nm) to 1.4 μm. It will be appreciated that any wavelength may be detected by an appropriate image capture means.

In yet a further embodiment, the device 10 may be adapted to be in a sleep mode when not active. The device 10 may then wake up from a sleep mode to acquire images in each colour or spectral band. The acquired data may be used to construct a 3D image from stereoscopic images and/or calculate various vegetation indices such as NDVI and PRI. The indices and/or the 3D data may be segmented to “bare ground”, and to different vegetation areas. The device software may allow for correlation of segmented images with 3D data in order to measure biomass and estimate biomass resource morphology. The device may further be adapted to calculate chlorophyll and nitrogen content in the detected vegetation using combination of different vegetation indices. The data generated may be reduced or compressed used to calculate statistical metrics. The results may then be sent to the server and the device 10 may then return to a sleep mode.

The device 10 may be adapted to include soil moisture probes which, in combination with the 3D image analysis of biomass resource growth stages and calculation of vegetation indices are used to predict at least one of; a biomass, a biomass resource yield and biomass resource requirements. In this way the predictive modelling capability of the device may be enhanced.

While the device 10 may be adapted to use near infrared for active illumination, the device 10 may be adapted to only detect and/or capture predetermined colours. Meteorological data may be transmitted to the device, or used in combination with the device 10, which may allow for projections or expected biomass data to be generated by the device or be used to determine whether there are times not to activate.

Optionally, a plurality of devices 10 may be positioned throughout a field, pasture or more generally a ‘location’ such that each device 10 can be adapted to monitor a portion of location. Each respective set of data generated or captured and stored by each respective device at a location may be used to generate a heat map or other visual representation to illustrate high yield portions of a location or field. In this way a farmer or carer of a location can focus on portions of the location for rehabilitation or locations which may need additional care without the need for providing additional resources for locations which may not be in need of additional resources. This may provide for a more economical tenure to a location which can reduce resource strain while also achieving an optimal yield from a location, such as an optimal biomass yield.

Further, drones or other aerial devices such as satellites may be used to capture further data to use in combination with the data captured by a device 10. The drones may be adapted to capture wind speeds, humidity data, images or any other desired data. The data obtained by the drones or aerial devices may be transmitted to at least one device 10, or may be transmitted or transferred to a server or other storage means to be used in combination with the data obtained by each device 10, meteorological or drone/satellite data. Preferably, the data captured by the device may be used to augment or drone or satellite data. The device 10 is preferably adapted to provide a high resolution and a high accuracy 3d measurement of a biomass resource, preferably with a high temporal rate, such as each hour, every day or any other desired temporal period. The drone or satellite data will typically be low accuracy and/or low resolution, or 2D images only, with a low temporal rate; such as several days, weeks, or months for example. As such, combining these data sets may allow for extrapolation or other data augmentations or predictions to be effected.

A time-of-flight camera (ToF camera) may be used by the device 10 or drone to capture 3D images. A ToF camera is a range imaging camera system that resolves distance based on the known speed of light, measuring the time-of-flight of a light signal between the camera and the subject for each point of the image. The time-of-flight camera is a class of scannerless LIDAR, in which the entire scene is captured with each laser or light pulse, as opposed to point-by-point with a laser beam such as in scanning LIDAR systems. These ToF cameras measure the direct time-of-flight required for a single laser pulse to leave the camera and reflect back onto the focal plane array. 3D images captured using this methodology image complete spatial and temporal data, recording full 3D scenes with single laser pulse. This allows rapid acquisition and rapid real-time processing of scene information. If a drone is used to capture this data captured the data may be augmented by the data collected by the device 10. Other 3D known scanning technology may be used by the device 10 or a drone or satellite to capture data and/or images related to a predetermined location.

In yet a further embodiment, at least one recorded image and/or data set is associated with an identification and/or a time stamp. The time stamp may include the local time of the recorded picture and the date. The identification may be a serial number or other assigned descriptor to distinguish devices 10. In another embodiment, the device 10 can be calibrated with marker plates, or reference plates. This may allow for a visual reference in captured images.

Identification of a harvestable resource, such as fruits, nuts and vegetables, may be achieved by a relative comparison between wavelengths emitted by the wavelength emitting means 108 and detected by the sensor 150 of the device. Further, as colours of harvestable resources may change during growth, or if they are a particular species which can cause difficulties with identification of foliage and a biomass resource. For example, identification of a red apple on an apple tree may require a different wavelength than identification of a green apple as the colours of the foliage of the tree may make it difficult for conventional devices to identify between the green of the biomass resource and the green of the foliage. A spectral signature of the biomass resource will generally be different than that of the foliage, and therefore determining the spectral signature can assist with identification of the harvestable resource on the biomass resource. The biomass signature may also provide additional information with respect to the growth stage of the biomass resource, and assist with determining when to provide fertilizers and pesticides. Detection of growth stages may assist with a reduction of fertilizer and pesticides used for a biomass resource as accurate predictions can be generated by the device.

Identifying spectral signatures may allow for a high contrast between a biomass resource and a further object in a captured image. For example, a harvestable resource may have a high contrast relative to the biomass resource, which allows for identification of a number of harvestable resources, and potentially a volume and/or density of harvestable resources.

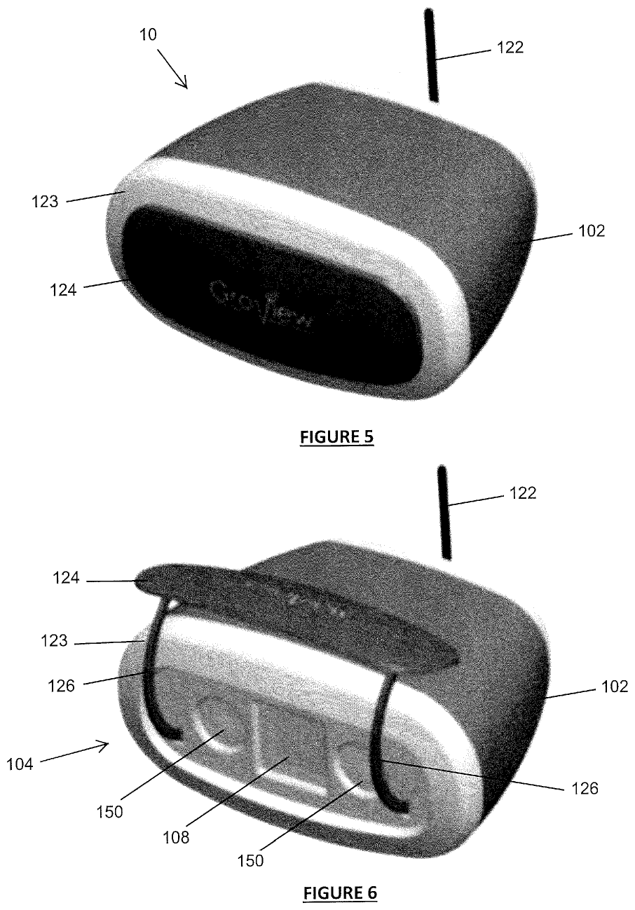

As shown in FIGS. 5 and 6, the device 10 has a housing 102 with a face portion 123. The face portion 123 having a plurality of apertures for the sensor 150 and wavelength emitting device 108. Each of the apertures further has a lens, or transparent element extending across the apertures to protect the sensors 150 internal the housing. Alternatively, the lens of the capture sensor 150 extends through an aperture. A lens cover 124 may be provided to cover the sensor 150 and wavelength emitting device. The lens cover is adapted to move from a covering position to an open position. The covering position protects or shields the lenses of the sensor 150 and the wavelength emitting means, and the open position allows for the image capture sensor 150 to capture images, and the wavelength emitting means 108 can emit light to a target biomass resource and/or harvestable resource.

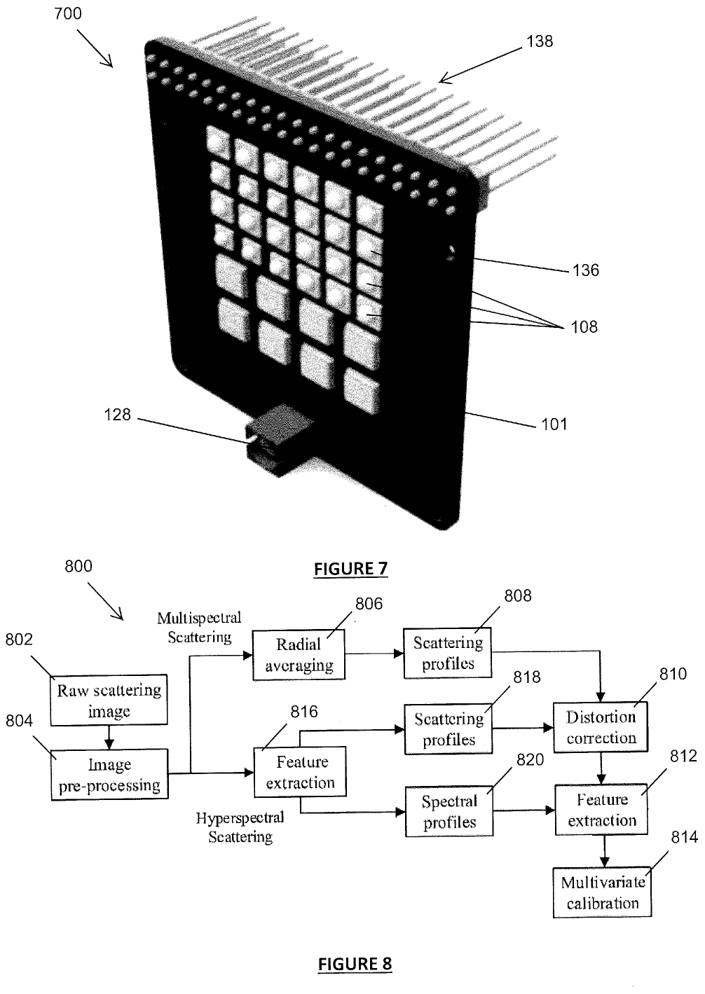

Referring to FIG. 7, there is illustrated a wavelength emitting device 700, which may be used as the wavelength emitting device 108. The wavelength emitting device 700 comprises a PCB 101 with a plurality of LEDs and a laser 128. The LEDs are disposed in a grid array which allows for desired LEDs to be activated for detection of a biomass resource. In one embodiment, the wavelengths emitted by the array of LEDs are preferably in the range of 400 nm to 1100 nm Exemplary wavelengths emitted may be around; 450 nm, 520 nm, 590 nm, 630 nm, 660 nm, 730 nm, 780 nm, 810 nm, 850 nm, 880 nm, 940 nm, and 980 nm The number of channels of the wavelength emitting device 108 may be in the range of 6 to 36 channels, however in this embodiment, the wavelength emitting device 108 comprises twelve (12) channels which emit light in the spectrum range of blue to NIR. A high power short pulse driver may also be provided to allow for capture of images when the lens cover 124 is lifted. Further, the pulse/camera synchronisation can be adapted to activate when the cover 124 is in a predetermined position, such as an open position. The cover 124 is shown being connected to a pair of arms 126 which move the cover from a first position to a second position. The first position can be a closed position, and the second position can be an open position. At least one arm 126 may be mounted to a motor internal the housing 102 such that activation of the motor can effect movement of the arm, and therefore the cover 124.

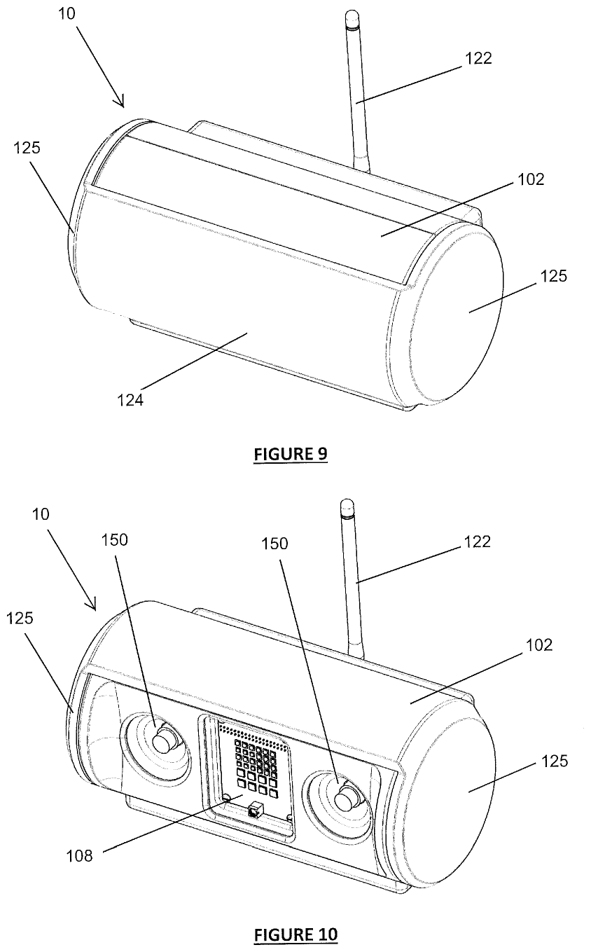

Referring to FIGS. 9 and 10, there is illustrated yet another embodiment of a device. FIG. 9 shows the device with a cylindrical housing 102 with a lens cover 124 in a first position, and FIG. 10 shows the lens cover in a second position. The lens cover 124 may be mounted on rails or tracks, or the lens cover 124 may be rotated about a central pivot location relative to the housing 102. If the cover rotates about a pivot location (not shown) the cover 124 may have a pair of side walls 125 which are connected to the pivot location. Having a pivotable lens cover 124 may shield the lens cover movement means and associated actuators and reduce the potential for the lens cover actuators to be damaged. The cylindrical housing 102 may also assist with wind and rain passing over the housing 102. Similar image capture sensor 150 and wavelength emitting device 108 may be used with the device 10 of this embodiment, similar to other embodiments described herein. A motor and actuator may be installed in the housing 102 of the device to move or otherwise actuate the lens cover from a first position to a second position.

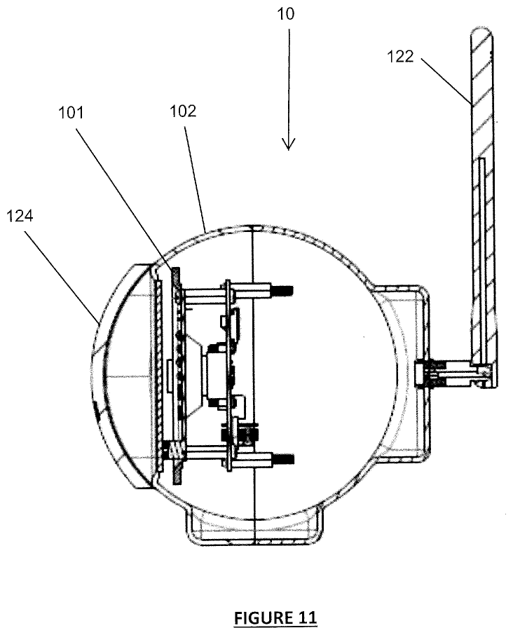

FIG. 11 shows a sectional side view of the embodiment as shown in FIGS. 9 and 10. The electronics PCB 101 is mounted in the housing 102 and the antenna 122 is mounted on the rear of the housing 102. While the antenna is not shown as being connected via wires to the PCB 101, the antenna 122 may be connected via any suitable conduit or conductive wires. The housing 102 walls are provided with securing means to allow for the PCB 101 to be mounted thereto.

The PCB 101 may also have a plurality of pins 138 which allow for powering the wavelength emitting devices and/or transferring data. A plurality of apertures 136 may also be disposed in the PCB 101 which allows for a securing means to mount therein to secure the PCB 101 in a desired location in the housing 102.

The LEDs may be adapted to activate in any predetermined order, with the predetermined order being configured with respect to the biomass resource being monitored. A laser 128 or group of lasers is also shown below the grid array of wavelength emitting means. The plurality of pins 138 disposed on the rear of the PCB 101 which are connectable to the electronics of the device 10 and can be used to communicate data to the processor, and to the storage device. Optionally, a low bandwidth communication means may be used to transmit or communicate data to a user device, such as a personal computer. The personal computer may be used to monitor all devices which are being used to monitor crops.

The laser 128 may be used to generate active 3D imaging using conventional methods. The laser 128 may be adapted to function as a “dot projector” which projects an array of laser dots on surfaces to determine distance and determine objects in view, relative to the laser 128. Using active 3D imaging in combination with spectral imaging can generate an image of the biomass and at least one property of the biomass resource.

The device 10 is capable of determining at least one attribute of a biomass resource. For example, the device may be used to inspect, grade and identify horticultural and food products (biomass resource) based on their spatial surface features such as shape, size and colour, a growth stage and the presence of surface defects. Further, the device may be used for identifying internal quality attributes, which may be chemical and/or physical, and in another embodiment may also detect defects.

Hyperspectral imaging, broadly speaking, is a type of spectral imaging technology that integrates imaging and spectroscopy to obtain 3D data cubes, which contain 2D spatial and one-dimensional (1D) spectral information from products. It is generally acknowledged that imaging at fewer than 10 discrete wavelengths may be referred to as multispectral, while imaging at more than 10 contiguous wavelengths or narrow wavebands is termed hyperspectral. Hyperspectral imaging provides a significant number of advantages over multispectral imaging, but it will be appreciated that the device 10 may be adapted to use both multispectral imaging and multispectral imaging.

Hyperspectral imaging may provide for detection of diseases and pests, detection of weeds, detection of nutrient deficiency (such as nitrogen, phosphorus and potassium), detection of nitrogen, yield prediction, biomass resource maturity, and general biomass resource health. Further, hyperspectral imaging may provide for an additional information collection relative to multispectral imaging. Multiple organisms may be identified via imaging and can be used to detect pests and weeds which may require the application of herbicides and/or pesticides to be applied to a crop (biomass resource).

The device 10 may capture hyperspectral image data via: point scanning, line scanning, area scanning and single shot. Point scanning captures spectral data for each pixel each time, and scans the full spatial scene by moving either the detector or the object continuously in the two spatial dimensions to obtain 3D image cubes. Spectral scanning methods can also be used in which the sample is generally stationary and scan with full spatial information is performed sequentially over a full spectral range. It will be appreciated that the device 10 may also be used to acquire a 3-D image cube of a large area with one exposure using hyperspectral imaging. Capturing a 3D image using this approach may provide for a fastest speed capture and subsequent analysis.

VIS to NIR, which is commonly used in hyperspectral imaging for food detection. LEDs may emit VIS to NIR wavelengths have evolved rapidly in recent years and become unstable output due to operating voltage fluctuations. LEDs are small size, fast response, low cost, long lifetime, low heat generation, and low energy consumption. In addition, lasers 128 may be used and have advantages which also include small size, fast response, low cost, long lifetime, low heat generation, and low energy consumption.

An embodiment of a flowchart 800 for processing of a captured image may be achieved by the processes as shown in FIG. 8. The raw scatter data captured 802 by the image is pre-processed 804 to remove data which is not needed for analysis. For example, pre-processing may remove data related to foliage of a biomass resource, and only retain data related to a biomass resource. The spectral data can be assessed based on the image capturing method. In this embodiment, the image capturing method is multispectral imaging, or hyperspectral imaging. Using multispectral imaging the radial average 806 of the scatter is calculated, and the scattering profiles 808 are generated. The scattering profiles may be indicative of at least one attribute. A distortion correction 810 can then be applied to the scatter profile(s) and features can be identified in the image, with commonly detected distortions being scattering distance distortion and/or intensity distortion. The features 814 of the image may be indicative of a growth stage of the biomass resource, and may show biomass resources growing on the biomass resource. Based on the data collected, the device may be recalibrated 814 for the next image capture.

With respect to hyperspectral image capture methods, features may be extracted 816 from the captured data. Based on the features in the data, scatter profiles 818 may be generated and/or spectral profiles may be generated. Similar to scattering profile processing for multispectral imaging, distortion corrections 810 may be applied and feature extraction 812 from the scatter profile can be performed. If the spectral profiles are generated, feature extraction may be performed without a distortion correction being performed. Again, using the features extracted, a multivariate calibration 814 may be performed. Alternatively, after feature extraction 816, spectral profiles 820 can be generated and feature extraction 812 of the spectral profiles may identify at least one spectral signature, or extract at least one feature of the spectral profiles. After features have been extracted, a multivariate calibration 814 may be performed.

Using the above image processing methods, a growth stage of a biomass resource and/or harvestable resource may be identified. If the biomass resource is at a key growth stage, for example a growth stage which requires pesticides to be added, the device 10 may issue an alert that a key growth stage of the biomass resource has been observed.

Raw scattering images captured by the capture means 104 of the device 10 may contain pixels of unexpected high or low intensities, compared to respective adjacent pixels, and may indicate abnormal or defective tissue spots at the surface of a biomass resource. These pixels may appear as dark spots in recorded images and can be removed when assessing the data captured.

Feature extraction from scattering profiles may use the mean and standard deviation (SD) of a scattering profile to reduce the profile to a predetermined number of parameters. The method is fast and simple to and uses relatively small computational effort. Alternatively, the scattering profile can be represented a mathematical function, such as a function selected from the following group; exponential, Gaussian, Lorentzian and Gompertz functions. It will be appreciated that other functions may also be used for identifying features in light scattering to generate scattering profiles. Parameters in these functions can be used as scattering features to characterise the scattering profiles to reduce the scattering profile to a predetermined number of features in the range of two (2) to ten (10) features, resulting in reductions in the raw data volume stored on the device 10. In addition to these distribution functions, a diffusion approximation model can also be used to extract optical properties from a scattering profile, such as absorption and scattering coefficients. Another method may be to apply image processing algorithms for feature extraction. Using these methods a growth stage of a biomass resource may be obtained.

Multivariate calibration can be used to build a quantitative model relating extracted scattering and spectral features with quality attributes of a target biomass resource when processing images. The multivariate calibration techniques can be linear and non-linear. Linear techniques, such as multiple linear regression (MLR), principal component regression (PCR) and partial least squares (PLS), are suited to model a linear relationships and can be used for prediction of quality attributes in a biomass resource; while non-linear techniques such as neural network (NN), convoluted neural network (CNN), and support vector machine (SVM) can be used for calibration purposes.

Hyperspectral imaging can be used to predict the firmness of a biomass resource. Further, hyperspectral imaging in the NIR spectrum may be used to predict or make an assessment in relation to SSC, firmness and growth stages of a biomass resource. Firmness of a biomass resource may be detected by using wavelengths in the range of 500 nm to 1000 nm via hyperspectral imaging. The acidity of biomass resource may also be calculated by the device 10 using spectral imaging methods.

In yet another embodiment, the device 10 can be used to monitor animals. Monitoring animals may be used to detect animals in heat, or whether animals are subject to disease and/or pests in an animal enclosure. A spectral signature may be used to differentiate animals at a location, and may also be used to determine if pest animals, such as foxes, are at a location. Spectral signatures may processed with respect to \with physical features observed such that more than one biomass resource species can be monitored.

The device 10 may also be used for mitigating risk, which may be of particular importance with respect to investment. Mitigating risk can also reduce the potential for funds to be unwisely used.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A spectral imaging device for determining a growth stage of a biomass resource, the device comprising;
one or two (stereoscopic) cameras and an active illumination means emitting a spectral wavelength;
the cameras are adapted to capture at least two images comprising data captured in the spectral wavelength emittable by the active illumination means;
a processor adapted to analyse at least one image; and
wherein images captured by stereoscopic camera generates a 2D and/or a 3D image.

2. The device as claimed in claim 1, wherein the active illumination means comprises at least one LED.

3. The device as claimed in claim 1 or claim 2, wherein a biomass index value can be generated with respect to the captured 2D and/or 3D image.

4. The device as claimed in any one of the preceding claims, wherein the device further comprises a communication means to transmit data.

5. The device as claimed in any one of the previous claims, further comprising a power source in communication with a renewable energy generator.

6. The device as claimed in any one of the previous claims, wherein the stereoscopic camera is a time of flight 2D and/or 3D camera.

7. The device as claimed in any one of the previous claims, wherein the device is adapted to record and store at least one 2D and/or 3D image.

8. The device as claimed in any one of the previous claims, wherein the device is adapted to detect nitrogen content via hyperspectral imaging.

9. The device as claimed in any one of the preceding claims, wherein a plurality of wavelengths are emitted by the at least one active illumination means in the range of 200 nm to 1000 nm.

10. The device as claimed in any one of the preceding claims, wherein the device detects at least one the height of a biomass resource; the shape of a biomass resource; the density of a biomass resource or any other predetermined property of a biomass resource.

11. The device as claimed in any one of the preceding claims, wherein the device is fixed to a pole and said device can rotate relative to said pole.

12. The device as claimed in any one of the preceding claims, wherein carotenoid pigment levels can be captured such that chlorophyll content of a biomass resource can be detected.

13. The device as claimed in any one of the preceding claims, wherein a Nitrogen content of a biomass resource can be detected.

14. The device as claimed in any one of the preceding claims, wherein the device further comprises a probe adapted to detect at moisture content of soil.

15. The device as claimed in claim 14, wherein the probe and in combination with the cameras analysis of biomass resource growth stages and calculation of vegetation indices is used to predict biomass, crop yield and biomass resource requirements.

16. The device as claimed in any one of the preceding claims, wherein biomass resource specific features can be detected by the device using active illumination spectroscopy.

17. The device as claimed in claim 16, wherein biomass resource feature is selected from the group of; a biomass resource height, a biomass resource growth stage, a number of shoots, plant attributes, flower attributes, fruit attributes, chlorophyll content, sugar level, acidity, vitamin C level, firmness, nitrogen content, and a colour of the biomass resource.

18. The device as claimed in any one of the preceding claims, wherein at least one of satellite data and drone data can be used to augment data captured by the device.

19. The device as claimed in any one of the preceding claims, wherein data captured by the device is transmitted to a server for analysis and presentation to a user.

20. The device as claimed in any one of the preceding claims, wherein a growth rate of a biomass resource is determined with respect to at least two 2D and/or 3D images captured with respect to the time period between the 2D and/or 3D captured images.
